(19) United States
(12) Patent Application Publication
(10) Pub. No.: US 2026/0227323 A1
(43) Pub. Date: Aug. 6, 2026

(54) IMPROVING LIMITING ERRORS IN COARSE SPECTRAL RESOLUTION REMOTE SENSING ESTIMATES OF GAS EMISSIONS FROM A GROUND LOCATED POINT SOURCE

(71) Applicant: GHGSAT INC., Montreal (CA)

(72) Inventors: Dylan JERVIS, Montreal (CA); Mathias STRUPLER, Montreal (CA); Jason MCKEEVER, Montreal (CA); Antoine RAMIER, Montreal (CA); Jean-philippe MACLEAN, Montreal (CA)

(21) Appl. No.: 19/154,601

(22) PCT Filed: Feb. 9, 2024
(86) PCT No.: PCT/CA2024/050173

§ 371 Date: Aug. 7, 2025

Related U.S. Application Data
(60) Provisional application No. 63/484,357, filed on Feb. 10, 2023.

(51) Int. Cl. G01N 21/3504; G01N 33/00

(52) U.S. Cl. CPC G01N 21/3504; G01N 33/0036

(57) ABSTRACT

Systems and methods for use in improving estimates of gas emissions from a ground located point source. A signal band of spectral data for the gas emissions is gathered from the ground located point source and for an upper reference band and for a lower reference band. The upper reference band consists of spectral data that has a wavelength greater than the signal band while the lower reference band consists of spectral data that has a wavelength that is lower than the signal band. Albedo measurements for the upper and lower reference bands can be used to estimate the albedo for the signal band and the estimated albedo can be used to compensate/correct for albedo related errors in the estimates for the gas emissions. Multiple spectral measurements of the same ground location are used to average down the random errors that place a floor on the achievable measurement precision.

### TECHNICAL FIELD

The present invention relates to spectrometry. More specifically, the present invention relates to systems and methods for improving estimates of gas emissions from a ground located point source.

### BACKGROUND

The climate change crisis of the first few decades of the 21st century has highlighted the need for pinpointing sources of materials that contribute to or exacerbate climate change conditions. Greenhouse gases such as methane and carbon dioxide must be minimized to alleviate the ever-approaching point of no return for climate change. To this end, satellite mounted cameras as well as aircraft mounted systems have been used to determine the sources of such substances using solar backscattered radiation.

Such systems measure the gas density along a path, or column, that a solar photon takes from the sun, through the atmosphere, reflected off the earth, and to the optical instrument mounted on the satellite or aircraft.

As is well-known, light in a specific spectral or wavelength band is gathered from a ground located point source. By judiciously selecting the wavelength band such that absorption features for the gas of interest are within the wavelength band, emission concentrations of that particular gas from a specific ground located point source can be estimated from the absorption features.

Unfortunately, errors in estimating the emission concentrations from these systems can occur. A major source of error is due to the spatial and spectral heterogeneity of the Earth's surface reflectance. This is especially the case for measurement systems with coarse spectral resolution.

The measurement error floor in these systems is set by a combination of the photon “shot noise” level and the number of independent measurements of the spectra. Such error is random in nature and limits the measurement precision in the absence of errors due from albedo variations.

There is therefore a need for systems and/or methods that can minimize such errors described above in emission concentration estimates.

### SUMMARY

The present invention provides systems and methods for use in improving estimates of gas emissions from a ground located point source. A signal band of spectral data for the gas emissions is gathered from the ground located point source. In addition, spectra data for an upper reference band and for a lower reference band is also gathered from the ground located point source. The upper reference band consists of spectral data that has a wavelength that is greater than the signal band while the lower reference band consists of spectral data that has a wavelength that is lower or lesser than the signal band. Albedo measurements for the upper and lower reference bands can be used to estimate the albedo for the signal band and the estimated albedo can be used to compensate/correct for albedo related errors in the estimates for the gas emissions.

In a first aspect, the present invention provides a method for gathering data for use in imaging spectrometry, the method comprising:
gathering light in a first specified wavelength band, said first specified wavelength band being from λ1 to λ2, λ2 being greater than λ1;
gathering light in a second specified wavelength band, said second specified wavelength band being from λ3 to λ4, λ3 being greater than λ1 and λ4 being greater than λ3;
gathering light in a third specified wavelength band, said third specified wavelength band being from λ5 to λ6, λ1 being greater than λ5 and λ6 being greater than λ5;
wherein

all of said light gathered is light from a specific ground located point source;
said light gathered is for use in estimating emissions of a specific from said point source;
said first specified wavelength band contains absorption features of said gas;
λ1, λ2, λ3, λ4, λ5, and λ6 are wavelengths of light.

In a second aspect, the present invention provides a method for use in imaging spectrometry, the method comprising:
gathering light in a first wavelength band, a second wavelength band, and in a third wavelength band, said first wavelength band being between said second and said third wavelength bands;
estimating a reference albedo for said second and third wavelength bands;
using said reference albedo to estimate an albedo for said first wavelength band;
using said albedo for said first wavelength band to correct for errors in estimates for emissions of a specified gas from a ground located point source;
wherein
said first specified wavelength band contains absorption features of said specified gas.

In a third aspect, the present invention provides a method for reducing random errors in imaging spectrometry, the method comprising:
gathering multiple spectral measurements of a ground located point source;
using said multiple spectral measurements to average spectral data gathered from said ground located point source to thereby reduce effects of random errors in said data.

In a fourth aspect, the present invention provides a system for gathering data for use in imaging spectrometry, the system comprising:
an optical signal splitter for splitting an incoming spectral data signal into two paths;
a reference bandpass filter receiving a split spectral data signal that has been redirected by said splitter into a first path of said two paths, said reference bandpass filter being for filtering said spectral data signal such that a sum of an upper reference spectral band and a lower reference spectral band is allowed through said reference bandpass filter;
a signal bandpass filter receiving said split spectral data signal that has been redirected by said splitter into a second path of said two paths, said signal bandpass filter being for filtering said spectral data signal such that a signal spectral band is allowed to pass through said signal filter;
a reference spectral data detector receiving said sum of said upper reference spectral band and said lower reference band, said reference spectral data detector being for detecting relevant spectral data from said sum;
a signal spectral data detector receiving said signal spectral band, said signals spectral data detector being for detecting relevant signal spectral data from said signal spectral data band;
wherein
said signal spectral data is for use in estimating gas emissions from a ground located point source;
said incoming spectral data signal is derived from said ground located point source.

### BRIEF DESCRIPTION OF THE DRAWINGS

The embodiments of the present invention will now be described by reference to the following figures, in which identical reference numerals in different figures indicate identical elements and in which:

FIG. 1 is a diagram detailing a spectral region and three selected bands;

FIG. 2 is a schematic spectral region showing the three selected bands and the reflected spectra;

FIG. 3 is an illustration showing multiple 2D images captured during one pass of a remote vehicle overflying a ground located point source;

FIG. 4 is a schematic diagram of a possible implementation of one aspect of the present invention;

FIG. 5 is a schematic diagram of another possible implementation of another aspect of the present invention.

### DETAILED DESCRIPTION

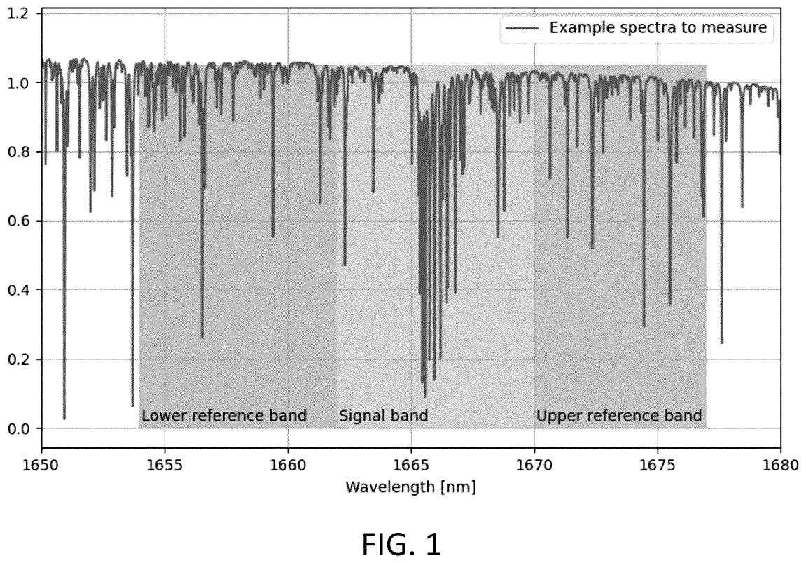

Referring to FIG. 1, illustrated is a spectral region showing three spectral bands. As can be seen, the first spectral or wavelength band 10 is between a second wavelength band 20 and a third wavelength band 30. The first wavelength band can be considered the signal band and preferably contains the absorption features of the gas of interest. The wavelength band can span from a wavelength of λ1 to a wavelength of λ2, with λ2 being greater than λ1. The second wavelength band can be considered the upper reference band is consists of wavelengths that are generally greater than λ1. For clarity, we can consider that the second wavelength band consists of wavelengths from λ3 to λ4, with λ4 being greater than λ3 and λ4 being greater than λ2. For the third wavelength band, this can be considered the lower reference band and consists of wavelengths that are generally less than λ1. For this lower reference band, this can consist of wavelengths from λ5 to λ6, with λ6 being greater than λ5 and λ1 also being greater than λ5.

In one aspect, the present invention provides for gathering light from the three spectral bands schematically illustrated in FIG. 1. Albedo related data can then be derived from both the upper and the lower reference bands and, using this albedo related data, the albedo for the signal band can be estimated. The estimated albedo for the signal band can then be used to correct for albedo related errors in the estimates for gas emissions from the ground located point source. As noted above, all the light for the various bands are gathered from the ground located point source. It should be clear that the signal band is selected such that it contains absorption features for the gas of interest.

It should also be clear that, while the three wavelength bands are shown in FIG. 1 as forming a continuous band, other options are possible. There may be a gap between the lower reference band and the signal band. Similarly, there may be a gap between the upper reference band and the signal band. As well, FIG. 1 shows that each of the bands is separate and distinct from the other bands. However, there may be overlap between either or both reference bands and the signal band. In addition, while the signal band contains absorption features for the gas of interest, it is preferred that the reference bands have minimal absorption features for this gas of interest. As can be seen from FIG. 1, the main absorption features are in the signal band but that there are absorption features in the reference bands. For best results, the number of the absorption features in the reference bands is, preferably, minimized.

It should be noted that, in the event there is a gap between a reference band and the signal band, it is preferred that the gap be minimized. Additionally, should the gap exist, it is preferred that the width of the gap be lesser than the width of either the reference band or the signal band. As an example, if the signal band has a width of approximately 10 nm, a gap between the signal band and a reference band should be, preferably, only a few nanometers in width.

It should also be noted that, in the event there is an overlap between the signal band and a reference band, the overlap should not appreciably include/cover the absorption features in the signal band. Additionally, it is preferred that any overlap be minimized. As an example, if the signal band has a width of approximately 10 nm, any overlap with a reference band should only be in the order of a few nanometers in width.

In terms of the width of the reference bands, it is preferred that each reference band is as wide as the signal band. Note, however, that this is not required for the present invention. As can be seen in the example in FIG. 1, the upper reference band has a different width from the lower reference band and that the signal band has a different width from either of these reference bands.

It should, however, be noted that it is preferable that the size of the different bands be approximately similar. That is, it is preferred that the reference bands be approximately the same or similar size to the signal band. In the event that the reference bands and the signal band are of different sizes/widths, it is preferred that these bands be within 25% of one another in size. That is, it is preferred that the size variation between the bands be, at most, approximately 25%.

It should also be clear that, as is known, the spectral data from the three wavelength bands is collected by way of a 2D camera detector array and that, accordingly, an image of the ground located point source is formed.

The inclusion of the reference bands in the data gathering is that the information in these reference bands can be used to account for any linear spectral variation in the earth's reflectance (i.e. spectral dependence of the albedo) in the local spectral region.

As can be imagined, the spectral bands are selected to optimize sensitivity to the gas of interest. A rule-of-thumb is to select a signal band that tightly envelopes the absorption features of the gas of interest and then selecting upper and lower reference bands that have as few features in them for the gas of interest. In FIG. 1, the gas of interest is methane and the closely spaced absorption features for methane can be seen to dip and cluster 1666 nm.

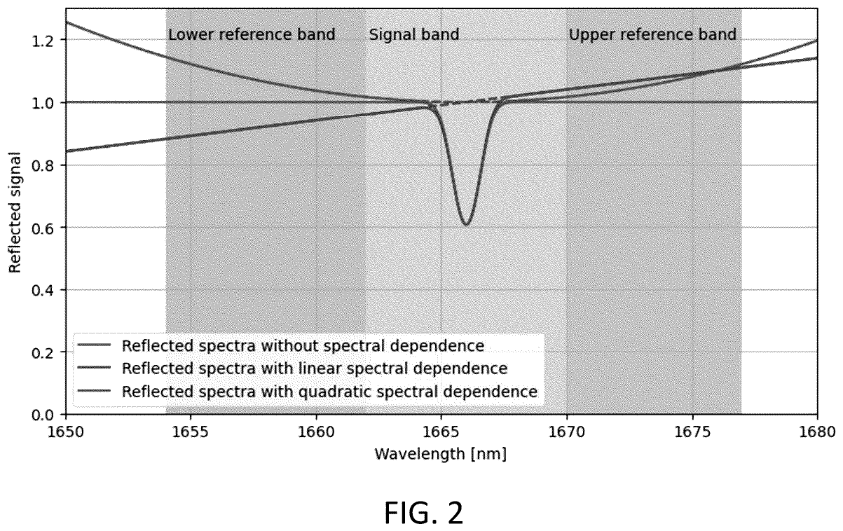

To explain the advantage obtained by including data gathering from upper and lower reference bands, FIG. 2 is provided. In FIG. 2, the example shows reflected signals for three surfaces that exhibit different spectral properties: (red) no spectral dependence of albedo, (blue) linear spectral dependence of albedo, and (green) quadratic spectral dependence of albedo. The dashed lines show the respective spectra in the absence of molecular absorption.

To explain the advantage of the present invention, it should first be noted that the reflectance of the Earth's surface is spectrally dependent. That is, the Earth reflects more incident light at some wavelengths than others. Additionally, it is known that different surfaces (e.g. desert surface, forest surface, and road surface) have different spectral properties.

When estimating the column density of the gas of interest (i.e., estimating the density of a column of gas emanating from the ground located point source of interest), one needs an estimate of the light signal magnitude in the absence of the gas (i.e. the dashed lines in FIG. 2).

Previously, an assumption was made that the albedo in one spectral band is the same as in the other. This was done if two spectral bands were sampled, with one band having more molecular absorption sensitivity than the other. Thus, for two spectral bands, the assumption is that the albedo for both bands is the same. However, whenever this assumption is broken—which is often, especially in scenes with multiple different surface types present—an error will result in the gas column density estimate. This albedo-correlated error can be very large and, in practice, limits the performance of multispectral gas measurements.

The present invention addresses this issue by allowing one to account for local, linear variation in the albedo's spectral dependence. As the present invention uses two reference bands, one above and one below the signal band in terms of wavelength, this allows one to measure a continuum of the albedo at two different spectral locations. This, therefore, allows one to infer the “slope” of the albedo's spectral variation. This information can then be used to infer what the albedo is in the signal band, thereby mitigating any artefacts arising from linear spectral variation.

As noted above, the use of the estimated albedo from the reference band allows for correction or at least the mitigation of errors in the gas emission estimates.

Such mitigation can be quantitatively estimated as shown in the following example. For this example, assume that the absorption signal changes by 5% for a 100% change in the gas being estimated. These values are typical for methane. If the albedo changes linearly by 1% between the signal band and one of the reference bands and this spectral variation is unaccounted for in the methane estimate, a (1%/5%)*(100%)=20% error will result. Such an error is large compared to the ideal photon shot-noise error of less than 1% of the system. However, such an error can be accounted for by using the present invention.

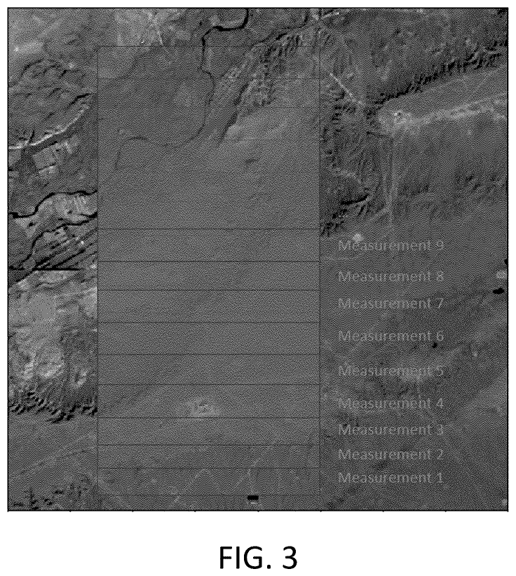

The limiting issue with imaging spectrometry in the absence of systematic error is random noise. In another aspect, the present invention addresses the issue of random noise by allowing one to reduce the effect of random noise in spectrometry datasets. Referring to FIG. 3, provided is an illustration of multiple 2D images (blue rectangles) captured for a single specific ground location. By taking multiple 2D images and generating N (or multiple) datasets for the same ground location, random noise can be averaged down by a factor of VN.

It should, however, be clear that N, the number of measurements, is determined by a combination of the image exposure time, the speed or panning of the mobile platform in which the measuring instrument is housed, and the field of view of the instrument. Typical instrument and mobile platform configurations would give N≈200, although this can be adjusted to trade performance for coverage (for a given amount of image data).

It should be clear that the multiple measurement aspect of the present invention allows for the lowering of the fundamental photon shot-noise error floor. This renders this aspect to be quite useful in the field of spectrometry.

In terms of implementation of the present invention, one option would be to route the data gathered from each point source through three bandpass filters, with each filter being configured to filter one of the bands of interest. Thus, one filter would only allow the signal band to pass while each of the other two filters would allow either the upper reference band or the lower reference band to pass.

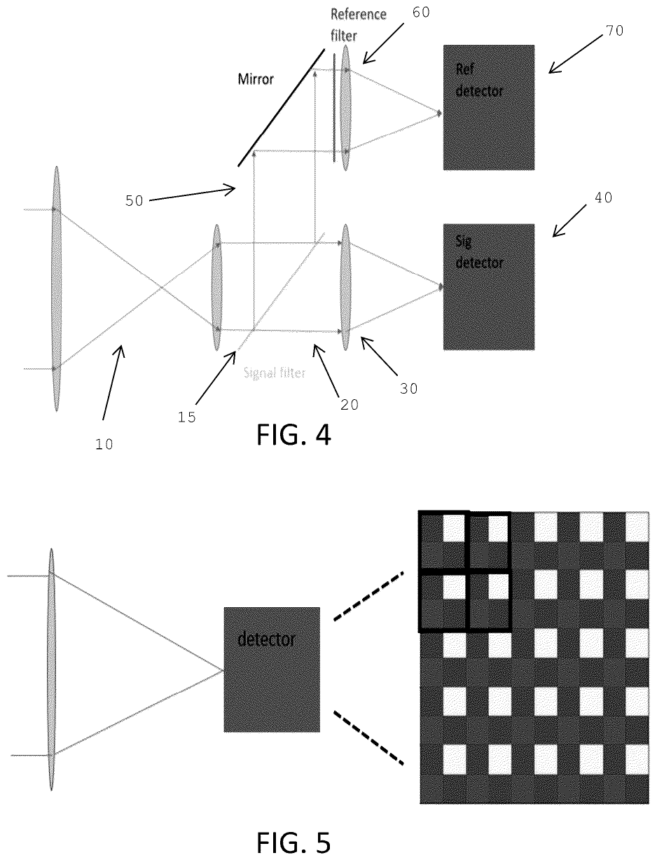

The above solution may, of course, be considered wasteful. Another implementation is schematically illustrated in FIG. 4. As can be seen from FIG. 4, the incoming spectral data 10 is split into two paths by way of an optical splitter 15. The first path 20 leads to a bandpass filter 30 that is configured to let the signal band pass through. The filtered signal band spectral data is then received by a signal detector/camera 40 that detects the relevant absorption features. The second path 50 leads to another bandpass filter 60 (a reference filter) that only allows the upper and the lower reference bands (or the sum of these two bands) to pass through. The spectral data from the upper and lower reference bands are then received by another signal detector/camera 70 that detects the relevant data from the upper and lower reference bands. The desired data from the upper and lower reference bands (in this case the albedo for these bands) can be extracted and then used in the processing of the absorption data from the signal band.

Another implementation is illustrated schematically in FIG. 5. This implementation uses a single camera design. This implementation places optical filters on individual pixels of a 2D detector array, enabling pixel-dependent spectral response. For this implementation, the detector array is organized into a series of 2×2 super-pixels, detailed as a 2×2 square in FIG. 5 with each super-pixel having two blue boxes, one red box, and one yellow box. In this configuration, two of the pixels measure light from the signal band (the blue boxes), one of the pixels measures light from the lower reference band (the yellow box), and one of the pixels measures light from the upper reference band (the red box).

In this 2×2 super-pixel-based implementation, the optical image of the ground located source point would be blurred (via choice of the optical point-spread function) to have at least a 2-pixel resolution in order to “smear out” signal variations due to spatial albedo inhomogeneity within the super-pixel. If, after the first gas retrieval, it is found that the gas measurement is corrupted by the heterogenous albedo, an additional gas retrieval step can be performed that makes use of the albedo estimate from the first retrieval.

It should be made clear that the various aspects of the present invention may be used in the image spectrometry estimation of any of the following gases: methane (CH₄), CO₂, H₂O, SO₂, NO₂, CO, O₂, and O₃.

A person understanding this invention may now conceive of alternative structures and embodiments or variations of the above all of which are intended to fall within the scope of the invention as defined in the claims that follow.

We claim:

1. A method for gathering data for use in imaging spectrometry, the method comprising:
gathering light in a first specified wavelength band, said first specified wavelength band being from λ1 to λ2, λ2 being greater than λ1;
gathering light in a second specified wavelength band, said second specified wavelength band being from λ3 to λ4, λ3 being greater than λ1 and λ4 being greater than λ3;
gathering light in a third specified wavelength band, said third specified wavelength band being from λ5 to λ6, λ1 being greater than λ5 and λ6 being greater than λ5;
wherein all of said light gathered is light from a specific ground located point source;
said light gathered is for use in estimating emissions of a specific from said point source;
said first specified wavelength band contains absorption features of said gas;
λ1, λ2, λ3, λ4, λ5, and λ6 are wavelengths of light.

2. The method according to claim 1, wherein a wavelength spectrum from said λ5 to said λ4 is continuous.

3. The method according to claim 1, wherein a wavelength spectrum for each of said first, second, and third wavelength bands is continuous.

4. The method according to claim 1, wherein there is an overlap between said first specified wavelength band and said second specified wavelength band.

5. The method according to claim 1, wherein there is an overlap between said first specified wavelength band and said third specified wavelength band.

6. The method according to claim 1, wherein there is a gap between said first specified wavelength band and said second specified wavelength band.

7. The method according to claim 1, wherein there is a gap between said first specified wavelength band and said third specified wavelength band.

8. The method according to claim 1, wherein said second and said third wavelength bands are used to correct for albedo related errors in estimates for said emissions of said specified gas from said point source.

9. The method according to claim 1, further comprising
estimating a reference albedo for said second and third wavelength bands;
using said reference albedo to estimate an albedo for said first wavelength band;
using said albedo for said first wavelength band to correct for errors in estimates for emissions of said specified gas from said point source.

10. The method according to claim 1, wherein said specified gas is any one of methane (CH4), CO2, H2O, SO2, NO2, CO, O2, and O3.

11. A method for use in imaging spectrometry, the method comprising:
gathering light in a first wavelength band, a second wavelength band, and in a third wavelength band, said first wavelength band being between said second and said third wavelength bands;
estimating a reference albedo for said second and third wavelength bands;
using said reference albedo to estimate an albedo for said first wavelength band;
using said albedo for said first wavelength band to correct for errors in estimates for emissions of a specified gas from a ground located point source;
wherein
said first specified wavelength band contains absorption features of said specified gas.

12. The method according to claim 11, wherein a wavelength spectrum across all three wavelength bands is continuous.

13. The method according to claim 11, wherein a wavelength spectrum for each of said first, second, and third wavelength bands is continuous.

14. The method according to claim 11, wherein said specified gas is any one of methane (CH4), CO2, H2O, SO2, NO2, CO, O2, and O3.

15. (canceled)

16. A method for reducing random errors in imaging spectrometry, the method comprising:
gathering multiple spectral measurements of a ground located point source;
using said multiple spectral measurements to average spectral data gathered from said ground located point source to thereby reduce an effect of random errors in said data.

17. The method according to claim 16, wherein said method is used in estimating emissions of a specified gas, said specified gas being any one of methane (CH4), CO2, H2O, SO2, NO2, CO, O2, and O3.

18. The method according to claim 1, further comprising:
gathering multiple spectral measurements of said ground located point source;
using said multiple spectral measurements to average spectral data gathered from said ground located point source to thereby reduce an effect of random errors in said data.

19. The method according to claim 11, further comprising:
gathering multiple spectral measurements of said ground located point source;
using said multiple spectral measurements to average spectral data gathered from said ground located point source to thereby reduce an effect of random errors in said data.
